(19) United States
(12) Patent Application Publication
(10) Pub. No.: US 2004/0087790 A1
(43) Pub. Date: May 6, 2004

(54) Process of making n-heterocyclic bicyclic lactone compounds from ketoamides

(76) Inventors: Todd D. Nelson, Exton, PA (US); Carl LeBlond, Johnstown, PA (US); Jeffrey V. Mitten, King of Prussia, PA (US)

Correspondence Address: MERCK AND CO INC, P O BOX 2000, RAHWAY, NJ, 070650907

(21) Appl. No.: 10/695,048

(22) Filed: Oct. 28, 2003

Related U.S. Application Data
(60) Provisional application No. 60/422,701, filed on Oct. 31, 2002.

(51) Int. Cl. C07D 498/02

(52) U.S. Cl. 544/105; 540/560

(57) ABSTRACT

A process of preparing a compound of Formula I

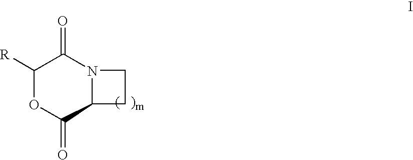

wherein

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino group; and

m is 1, 2, 3, 4, or 5.

### CROSS REFERENCE TO RELATED APPLICATIONS

This application claims the benefit of U.S. Provisional Application No. 60/422,701, filed Oct. 31, 2002.

### BACKGROUND OF THE INVENTION

Thrombin is a serine protease present in blood plasma in the form of a precursor, prothrombin. Thrombin plays a central role in the mechanism of blood coagulation by converting the solution plasma protein, fibrinogen, into insoluble fibrin. Thrombin inhibition is useful in treating and preventing a variety of thrombotic conditions, such as coronary artery and cerebrovascular disease, and preventing coagulation of stored whole blood or coagulation in other biological samples for testing or storage. Thrombin inhibitors can be added to or contacted with any medium containing or suspected of containing thrombin and in which inhibiting blood coagulation is desired, e.g., when contacting a mammal's blood with vascular grafts, stents, orthopedic prosthesis, cardiac prosthesis, and extracorporeal circulation systems. Those experienced in this field are readily aware of the circumstances requiring anticoagulant therapy.

Molecules that selectively inhibit the formation of thrombin or modulate the activity of thrombin have the potential to regulate many of the above-mentioned disease states. Proline-derivative thrombin inhibitor 1 is one such molecule. The known synthesis of this compound (see WO 02/50056) requires linear assembly using standard peptide coupling and multiple protection and deprotection manipulations.

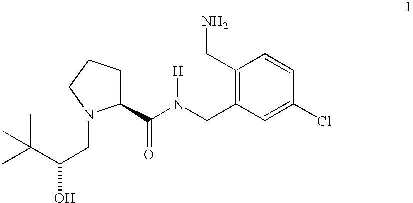

The present invention is an efficient process for preparing compound 1 and structurally related compounds.

### SUMMARY OF THE INVENTION

This invention is directed to synthesis of N-heterocyclic bicyclic lactone compounds of formula I, and its novel ketoamide precursors of formula IV,

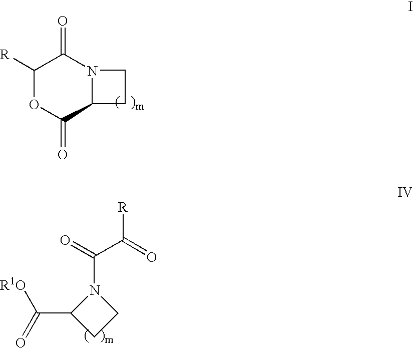

comprising coupling a keto acid of formula II with an ester of formula III:

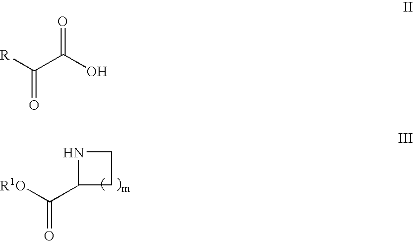

in the presence of a peptide coupling reagent, to produce the novel ketoamide of formula IV, wherein

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino group;

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen; and

m is 1, 2, 3, 4, or 5.

The novel ketoamide of formula IV may be reduced to a hydroxy amide, which may be cyclized in the presence of an acid to the N-heterocyclic bicyclic lactone compounds of formula I.

The invention also includes the novel ketoamide precursors of formula IV,

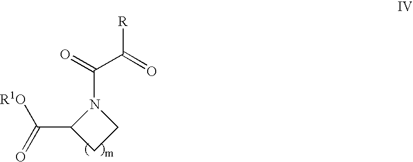

wherein

R is

a) C₁₋₆ alkyl unsubstituted or substituted with one, two, or three groups independently selected from C₆₋₁₀ aryl, C₁₋₆ alkoxy, halogen, and amino; or

b) 6-10 membered monocyclic or bicyclic aryl, unsubstituted or substituted with one, two or three groups independently selected from Cl ₆ alkyl, C₁₆ alkoxy, halogen, and an amino group;

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen; and

m is 1, 2, 3, 4, or 5.

The invention also includes methods of using the novel N-heterocyclic bicyclic compounds and its novel ketoamide precursors to make thrombin inhibitors.

The foregoing general description and the following detailed description are exemplary and are intended to provide further explanation of the claimed invention.

### DETAILED DESCRIPTION OF THE INVENTION

The invention is a process of preparing a compound of formula I

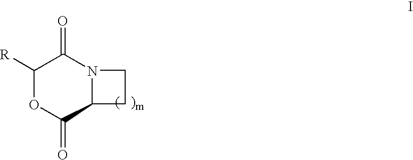

wherein

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino group; and

m is 1, 2, 3, 4, or 5;

comprising:

a) coupling a keto acid of Formula II,

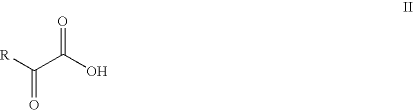

in presence of a peptide coupling reagent, with a compound of Formula III

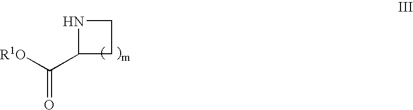

wherein

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen;

to produce a a ketoamide of forumla IV,

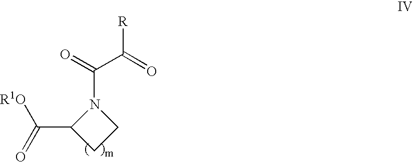

b) reducing ketoamide of formula IV to produce a hydroxyamide of Formula V,

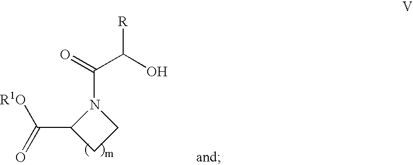

c) cyclizing the hydroxyamide of formula V in the presence of an acid to produce a compound of formula I.

In one embodiment of the process, R is an unsubstituted C₁₋₆ alkyl, e.g. tert-butyl. In another embodiment, R¹ is methyl. In another embodiment, m is 1. In another embodiment of the process, the peptide coupling agent is 1-ethyl-3-(3-dimethylaminopropyl)carbodiimide. In another embodiment of the process, the acid is p-toluene sulfonic acid.

Compounds of formulas (I)-(IV) above may have chiral centers and occur as racemic mixtures, individual diastereomers, or enantiomers, with all isomeric forms. The scope of the present invention includes individual enantiomers of compounds of formula (I)-(IV) as well as mixtures of enantiomers of compounds of formula (I)-(IV) in any proportion, including racemic mixtures. Generally it is preferred to use a compound of formula (I)-(IV) in the form of a purified single enantiomer, most preferably the (S) isomer.

When any variable occurs more than one time in any constituent or in formula I-IV, its definition on each occurrence is independent of its definition at every other occurrence. Also, combinations of substituents and/or variables are permissible only if such combinations result in stable compounds.

Compounds prepared according to the process of the invention are useful in preparing compounds that are useful for treating or preventing a variety of thrombotic conditions, including venous thromboembolism (e.g. obstruction or occlusion of a vein by a detached thrombus, obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and lowering the propensity of devices that come into contact with blood to clot blood.

Some abbreviations that may appear in this application are as follows:

### Abbreviations

Designation

| | |
|---|---|
| ACN | acetonitrile |
| Boc | tert butoxycarbonyl |
| DBU | 1,8-diazabicyclo[5.4.0]undec-7-ene |
| CH₃CN | acetonitrile |
| DCM | dichloromethane |
| DIEA | diisopropylethylamine |
| DIPEA | diisopropyethylamine |
| DMF | dimethylformamide |
| EDC | 1-ethyl-3-(3-dimethylaminopropyl)carbodiimide hydrochloride |
| EtOH | ethanol |
| HOBt | 1-hydroxybenzotriazole hydrate |
| IPAc | isopropyl acetate |
| IPA | isopropyl alcohol |
| MeOH | methanol |
| NaBH₄ | sodium borohydride |
| Na₂CO₃ | sodium carbonate |
| NMP | N-methyl pyrrolidinone |
| PTSA | p-toluenesulfonic acid |
| TEA | triethylamine |
| TFA | trifluoroacetic acid |
| THF | tetrahydrofuran |

Unless otherwise noted, the term “alkyl” includes both branched- and straight chain saturated aliphatic hydrocarbon groups having the specified number of carbon atoms for example, “C₁₋₆ alkyl” means an alkyl group having 1 to 6 carbon atoms, e.g., 1, 2, 3, 4, 5 or 6.” For illustration and not limitation, the alkyl may be methyl, ethyl, propyl, butyl, etc. The alkyl group may be unsubstituted or substituted with, for example, C₆₋₁₀ aryl, hydroxy, C₁₋₆ alkoxy, halogen, or amino.

Unless otherwise noted, “halogen”, as used herein, includes fluorine, chlorine, bromine, and iodine.

Unless otherwise noted, “alkoxy” means a linear or branched alkyl group of indicated number of carbon atoms attached through an oxygen bridge. “C₁₋₆ alkoxy” means any alkoxy having 1 to 6 carbon atoms, e.g., 1, 2, 3, 4, 5 or 6.

Unless otherwise noted, the term “aryl” includes a “C₁₋₆ alkoxy” means on alkoxy group having 6- to 10-membered mono- or bicyclic ring system such as phenyl, or naphthyl. The aryl ring can be unsubstituted or substituted with, for illustration and not limitation, one or more of C₁₋₆ alkyl; hydroxy; C₁₋₆ alkoxy; halogen; or amino.

Unless otherwise noted, the term “solvent” includes any polar solvent such as, for example, triethylamine, isopropyl alcohol, N-methyl pyrrolidinone, dimethylformamide, diisopropyethylamine, CH₃CN and tetrahydrofuran.

Unless otherwise noted, the term “acid” includes any acid such as a Bronsted Lawry or Lewis acid donating a proton or receiving an electron, which has a pH lower than 7.

Unless otherwise noted, the term “amino protecting group” includes, for illustration and not limitation, -C(O)OR, wherein R is any alkyl group such as C₁₋₄ alkyl.

Unless otherwise noted, “peptide coupling reagent” includes any class of compounds that mediate the coupling of an amine and a carboxylic acid such as, for example, 1-ethyl-3-(3-dimethylaminopropyl)carbodiimide hydrochloride.

For illustration and not limitation, an example of a keto acid of general formula II for use according to the invention includes tert-butyl-C(O)C(O)OH and pharmaceutically acceptable derivatives or solvates thereof.

The keto acid of formula II may be in the form of a purified single enantiomer, (S) or (R) isomer, or a mixture of both. The R group of formula II and R¹ group of formula III may be any designated members, respectively, independent of each other. For example, R may be t-butyl or any other alkyl or an aryl group regardless of whether R¹ is an alkyl, substituted or unsubstituted benzyl, or hydrogen. Similarly, R¹ may be an alkyl, substituted or unsubstituted benzyl, or hydrogen, regardless of whether R is an alkyl or an aryl group.

For illustration and not limitation, an example of an ester of formula III for use according to the invention is:

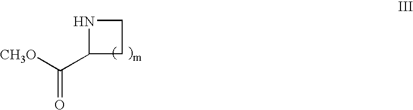

and pharmaceutically acceptable derivatives or solvates thereof. The ester of formula (II) may be in the form of a purified single enantiomer, (S) or (R) isomer, or a mixture of both. For illustration and not limitation, the ester of general formula III has been described with R¹ as CH₃. The R group of formula II and R¹ group of formula III may be any designated members, respectively, independent of each other. For example, R may be t-butyl or any other alkyl or an aryl group regardless of whether R¹ is an alkyl, substituted or unsubstituted benzyl, or hydrogen. Similarly, R¹ may be an alkyl, substituted or unsubstituted benzyl, or hydrogen, regardless of whether R is an alkyl or an aryl group.

For illustration and not limitation, an example of the novel ketoamide precursors of formula IV for use according to the invention is,

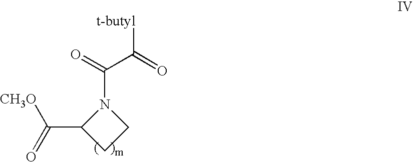

and pharmaceutically acceptable derivatives or solvates thereof. The ketoamide of formula (IV) may be in the form of a purified single enantiomer, (S) or (R) isomer, or a mixture of both. For illustration and not limitation, the ketoamide of general formula IV has been described with R as t-butyl and R¹ as CH₃. The R and R¹ groups of general formula IV may be any designated R and R¹ groups, respectively, independent of each other. For example, when R is t-butyl, R¹ may be an alkyl, including methyl, substituted or unsubstituted benzyl, or hydrogen. Similarly, when R¹ is a methyl, R may be an alkyl, including tert butyl, or an aryl group.

It will be appreciated by those skilled in the art that the compounds of formula I-IV may be modified to provide pharmaceutically acceptable derivatives thereof at any of the functional groups in the compounds of formula I-IV. Such derivatives are clear to those skilled in the art, without undue experimentation.

The pharmaceutically-acceptable salts of the compounds of the invention are prepared according to the procedures described herein and include those derived from pharmaceutically acceptable inorganic and organic acids such as e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

General Scheme I demonstrates, for illustration and not limitation, synthesis of N-heterocyclic bicyclic lactone of formula I and its novel ketoamide precursors of formula IV.

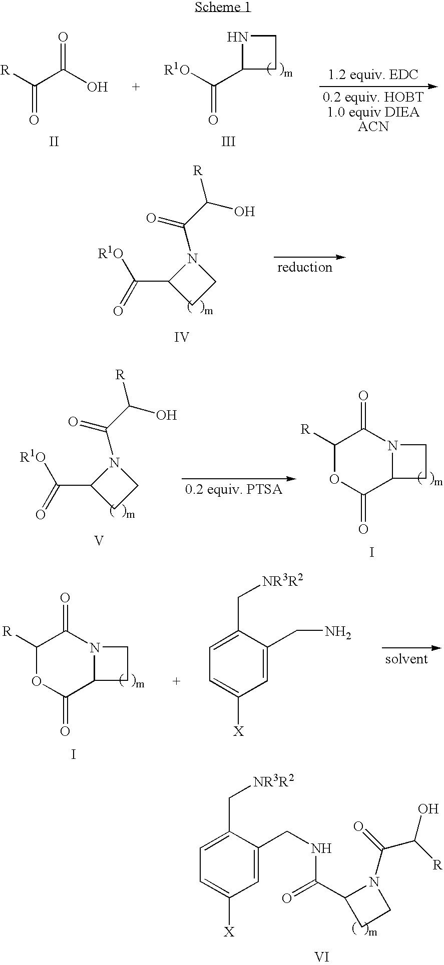

X is halogen, R² is an amino protecting group, R³ is hydrogen or an amino protecting group, and R, R¹ and m are as previously defined.

The inventive process comprises peptide coupling a keto acid of formula II, such as 1, with an ester of formula m attached to an N-based heteroalkyl ring, such as proline, to yield a novel ketoamide precursor, (S)-2. The carbinol stereochemistry may be created by a substrate-controlled asymmetric reduction of ketoamide (S)-2. Similar asymmetric reductions of I-keto proline amides and I, [symbol] -unsaturated proline amides have been reported to occur with low levels of stereochemical induction. (Munegumi, T.; Fujita, M.; Maruyama, T.; Shiono, S.; Takasaki, M.; Harada, K. BULL. CHEM. SOC. JPN. 1987, 60, 249; Boulmedais, A.; Jubault, M.; Tallec, A. BULL. SOC. CHIM. FR. 1989, 185; Byun, I. S.; Kim, Y. H. SYNTH. COMMUN. 1995, 25, 1963; Mallat, T.; Baiker, A. APPL. CATAL. A; GENERAL 2000, 200, 3. Prolinol-based ketoamides (hemiacetals) have been reduced with good stereoselectivity; Pansare, S. V.; Ravi, R. G. TETRAHEDRON 1998, 54, 14549.)

The following examples are for illustration and not limitation.

### EXAMPLE 1

An embodiment, for illustration and not limitation, of the synthesis of N-heterocyclic bicyclic lactone, (S, R)-5, and its novel ketoamide precursor, (S)-2, is the following:

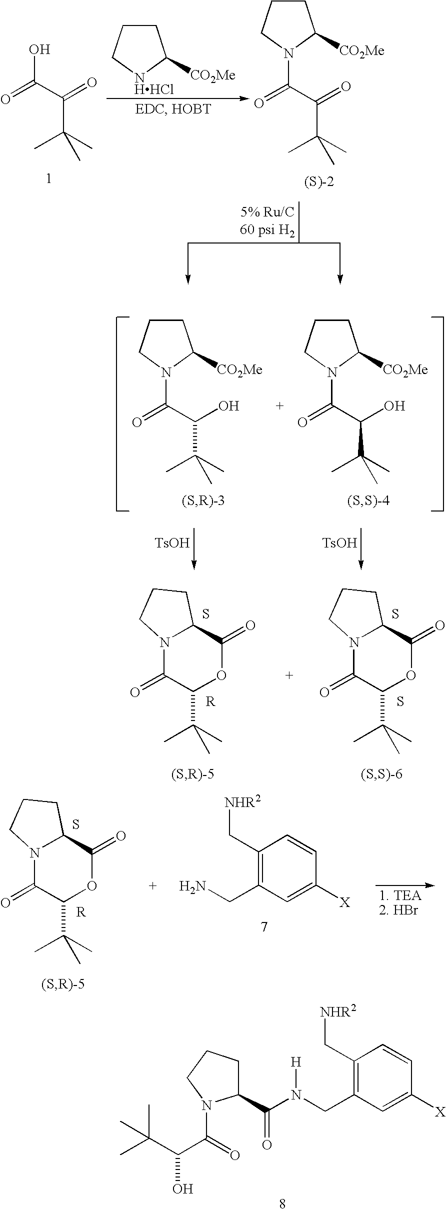

R² is an amino protecting group. X is halogen.

Step A: Preparation of (S)-2 ketoamide

Ketoamide (S)-2 was prepared in a single step (84%) by coupling keto acid 1 with L-proline methyl ester (EDC, HOBT). Various catalytic hydrogenation reducing agents such as Ru/C, Pt/C, Pd/C or Rh/C for the substrate-controlled reduction of the carbonyl group. The Pt/C, Pd/C or Rh/C hydrogenation catalysts resulted in decreased rate of carbonyl reduction. (Leading references for examples of 1-ketoamide reductions include: Ru-clay: Aldea, R. alper, H. J. ORG. CHEM. 1998, 63, 9425; Rh: Carpentier, J.-F.; Mortreux, A. TETRAHEDRON ASYMM. 1997, 8, 1083; Pt/Al₂O₃: Wang, G.-Z; Mallat, T.; Baiker, A. TETRAHEDRON ASYMM. 1997, 8, 2133; Ru: Chiba, T.; Miyashita, A.; Nohira, H.; Takaya, H. TETRAHEDRON LETT. 1993, 34, 2351.) The resulting mixture of diastereomeric hydroxy esters were lactonized with catalytic toluene sulfonic acid to afford a 93:7 ratio of lactones [(S,R)-5:(S,S)-6].

Step B: Preparation of hydroxy ester (S,R)-3 and (S,S)-4

Ketoamide (S)-2 (0.5 gm, 2.07 mmol), 5% Ru/C (0.25 gm) and methanol (50 ml) were combined and hydrogenated at 50° C. and 40 psig of hydrogen pressure. After 72 hours no ketoamide was visible by GC. The mixture was cooled to RT, depressurized and filtered through a celite packed sintered glass funnel. The methanol was removed in vacuo to yield the crude hydroxy ester (S,R)-3 and (S,S)-4.

Step C: Preparation of (S,R)-5 and (S,S)-6 lactones

The hydroxy ester was dissolved in 15 ml of toluene and PTSA (60 mg, 0.15 equivalents) was added. The mixture was stirred at room temperature and vacuum applied (40 mm Hg) to remove the methanol which is formed as a byproduct. After 3 hours, LC analysis indicated that the cyclization was complete. The ratio of (S,R)-lactone to (S,S)-lactone was 10:1. The toluene was removed in vacuo and the (S,R)-lactone purified by silica gel chromatography with 4:1 DCM:EtOAc to yield 293 mg of (S,R)-lactone (1.39 mmol, 67% yield from ketoamide, 98.4 LCAP).

The cyclization of hydroxy ester (S,R)-3 to lactone (S,R)-5 occurred more rapidly than the cyclization of the diastereomeric hydroxy ester (S,S)-4 to lactone (S,S)-6. An equimolar mixture of diastereomeric esters (S,R)-3 and (S,S)-4 were prepared by the EDC/HOBT peptide coupling of (rac)-hydroxy acid and L-proline methyl ester HCl. These esters were lactonized in toluene at 24° C., with 10% TsOH. The steady state formation of lactone (S,R)-5 occurred <2 hours. The molar rate of (S,R)-5 formation was more rapid than the diastereomeric lactonization of hydroxy ester (S,S)-4 to lactone (S,S)-6 [kᵣₑₗ=17]. The 5,6-fused ring system can exist in two conformations (A, B). For lactone (S,R)-5, conformer B is preferred.

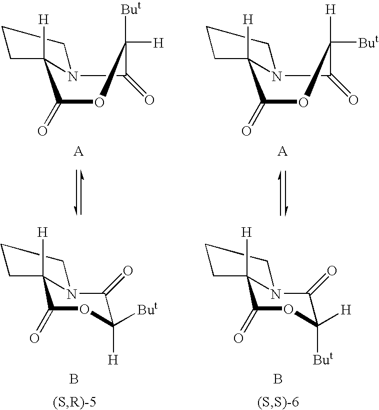

### EXAMPLE 2

Preparation of Compound 8

The coupling of the lactone (S,R)-5 with 7 (the preparation of which is described in WO 02/50056 on page 36) was accomplished in either TEA or IPA and occurred without the need for a catalyst or base additive. While many lactone aminolysis reactions require more rigorous conditions or are facilitated by the addition of catalysts, the facile nature of this amidation can be attributed to the inherent strain in lactone (S,R)-5.

Other polar solvents screened (NMP, DMF, DIPEA, CH₃CN, THF) resulted in slightly slower coupling rates or incompatibility with the lactone (MeOH, EtOH). The invention encompasses coupling the lactone with TEA or IPA or other polar solvents, such as NMP, DMF, DIPEA, CH₃CN, THF, as well as any other suitable reagents.

Alternatively, the lactone opening in THF could be accelerated by performing the reaction at 40° C. in the presence of HOAc. Regardless of the method for coupling the two fragments, the subsequent workup may include a wash with 2 M citric acid or similar reagent to remove amine 7 followed by a wash with 0.2 N NaOH or similar reagent (or saturated Na₂CO₃) to hydrolyze and remove unreacted lactone (S,R)-5. This process occurred without epimerization of either stereocenter. (S,R)-8 could be isolated as an amorphous solid following solvent removal, but this compound was typically utilized in the deprotection without isolation. Numerous conditions were screened for the unmasking of the benzyl amine by amino protecting group deprotection. Problems ranged from sluggish reactivity to product decomposition (spontaneous lactonization with extrusion of the corresponding diamine). The optimized conditions for protecting group removal incorporated the addition of a 6 wt % HBr (3 eq) solution to an anhydrous IPAc solution of the substrate. This afforded the target molecule HBr salt as a white amorphous solid in an overall 80% isolated yield from lactone (S,R)-5.

The above embodiment of the synthesis of N-heterocyclic bicyclic lactone compounds of formula (I) and its novel ketoamide precursors of formula (IV) is for illustration and not limitation. For example, a variety of keto acids of formula (II) may be coupled with a variety of esters of formula (III) using a variety of peptide coupling reagents to produce a variety of the novel ketoamides of formula (IV). The novel ketoamide of formula (IV) may be reduced to a hydroxy amide of formula (V) using a variety of reducing agents. The resulting hydroxy amide of formula (V) may be cyclized to a lactone of formula (I) using a variety of acids. Although lactone, (S,R)-5, was coupled with compound 7 using TEA or IPA or other polar solvents, such as NMP, DMF, DIPEA, CH₃CN, THF, to create compounds of formula (VI), other polar solvents may be used. The disclosed reagents, such as acids, peptide coupling reagents, and solvents, as well as amount of disclosed compounds may be modified by one of ordinary skill in the art without undue experimentation. The invention encompasses modification of the given ingredients, reagents, and ranges.

It will be apparent to those skilled in the art that various modifications and variations can be made to the synthesis of the N-heterocyclic bicyclic lactone and its novel ketoamide precursors of the present invention without departing from the scope of the invention. Thus, the present invention include modifications and variations that are within the scope of the claims and their equivalents.

What is clamed is:

1. A process of preparing a compound of Formula I 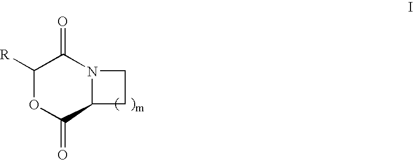
wherein
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino group; and
m is 1, 2, 3, 4, or 5;
comprising:
a) coupling a keto acid of Formula II, 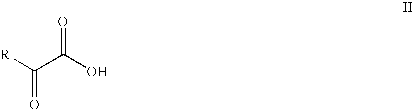
in presence of a peptide coupling reagent, with a compound of Formula III 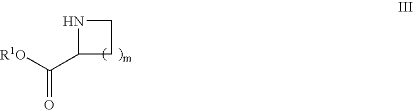
wherein
R¹ is
a) C₁₋₆ alkyl unsubstituted or substituted with one, two, or three groups independently selected from C₆₋₁₀ aryl, hydroxy, C₁₋₆ alkoxy, halogen, and amino;
b) benzyl unsubstituted or substituted with one, two or three groups independently selected from C₁₋₆ alkyl, hydroxy, C₁₋₆ alkoxy, halogen, and amino; or
c) hydrogen;
to produce a a ketoamide of Forumla IV, 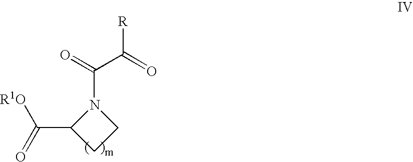
b) reducing ketoamide of Formula IV to produce a hydroxyamide of Formula V, 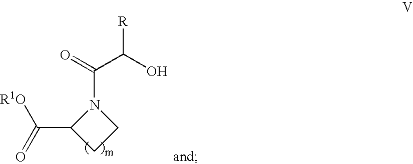
c) cyclizing the hydroxyamide of Formula V in the presence of an acid to produce a compound of Formula I.

2. The process of claim 1 wherein R is an unsubstituted C₁₋₆ alkyl.

3. The process of claim 2 wherein R group is tert-butyl and m=1.

4. The process of claim 1 wherein R¹ is methyl and m=1.

5. A compound of Formula IV or a pharmaceutically acceptable salt thereof, 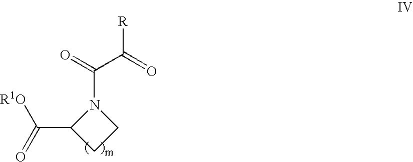
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino group;
R¹ is
a) C₁₋₆ alkyl unsubstituted or substituted with one, two, or three groups independently selected from C₆₋₁₀ aryl, hydroxy, C₁₋₆ alkoxy, halogen, and amino;
b) benzyl unsubstituted or substituted with one, two or three groups independently selected from C₁₋₆ alkyl, hydroxy, C₁₋₆ alkoxy, halogen, and amino; or
c) hydrogen; and
m is 1, 2, 3, 4, or 5.

6. A process of claim 1, wherein the peptide coupling agent is 1-ethyl-3-(3-dimethylaminopropyl)carbodiimide.

7. A process of claim 1, wherein the acid is p-toluene sulfonic acid.
